(19) United States
(12) Patent Application Publication
(10) Pub. No.: US 2026/0240536 A1
(43) Pub. Date: Aug. 20, 2026

(54) Knotless Constructs And Methods Of Tissue Repair

(71) Applicant: Arthrex, Inc., Naples, FL (US)

(72) Inventors: Timothy J. MCINTYRE, Naples, FL (US); Justin M. BOYLE, Naples, FL (US)

(73) Assignee: Arthrex, Inc., Naples, FL (US)

(21) Appl. No.: 19/544,355

(22) Filed: Feb. 19, 2026

Related U.S. Application Data
(60) Provisional application No. 63/760,686, filed on Feb. 20, 2025.

(51) Int. Cl. A61B 17/04

(52) U.S. Cl. CPC A61B 17/0401; A61B 2017/0445; A61B 2017/0464

(57) ABSTRACT

Surgical anchor constructs, fixation devices, and methods for tissue repairs are disclosed. A soft suture anchor has a cannulated sheath (that is fixed in bone), at least one repair suture attached to the sheath, and a plurality of flexible couplers attached to the sheath. At least one of the flexible couplers may be an anchor passing suture. At least one of the flexible couplers may be an anchor deployment suture.

### CROSS-REFERENCE TO RELATED APPLICATIONS

This application claims priority to U.S. Provisional Patent Application No. 63/760,686 filed Feb. 20, 2025, the disclosure of which is hereby incorporated by reference in its entirety herein.

### BACKGROUND

The disclosure relates to the field of surgery and, more specifically, to knotless anchor constructs and associated methods of tissue repairs.

### SUMMARY

Reconstruction systems, assemblies and methods for fixation of tissue are disclosed.

A surgical construct may create a knotless, self-locking, reinforced repair. A surgical construct may be made completely of suture to achieve fixation in bone without a separate anchoring body. A surgical construct may be employed in knotless fixation of first tissue to second tissue, for example, fixation of soft tissue to bone. A surgical construct may be a knotless, tensionable construct that includes a soft anchor in the form of an all-suture anchor. In some implementations, the soft anchor may include a soft anchor sheath with one or more repair sutures and at least two additional flexible strands. In some implementations, the one or more repair sutures may extend in a direction different from the direction of the at least two additional flexible strands. In some implementations, one of the additional flexible strands may be an anchor passing suture and another of the additional flexible strands may be an anchor deployment suture.

Methods of tissue repairs are also disclosed. In some implementations, knotless surgical constructs provide tissue fixation without any knot formation, by providing an all-suture repair which is conducted with fewer steps, increased anchor fixation and suture management, and increased reproducibility.

These and other features and advantages of this disclosure will become apparent and will be understood from the following detailed description of the various aspects of the disclosure taken in conjunction with the accompanying drawings.

### BRIEF DESCRIPTION OF THE DRAWINGS

FIG. 1 illustrates a surgical construct.

FIG. 2 illustrates another surgical construct.

FIGS. 3-13 illustrate schematic steps of an exemplary surgical repair with the surgical construct of FIG. 2.

FIG. 14 illustrates another view of surgical construct of FIG. 2.

### DETAILED DESCRIPTION

Tensionable, knotless, self-locking surgical constructs can create knotless, reinforced repairs.

A soft tissue repair system may include one or more knotless, tensionable constructs. In some implementations, a knotless, tensionable construct may include an all-suture anchor.

In some implementations, a knotless, tensionable construct may include a soft suture anchor with (i) one or more repair sutures (flexible strands) attached to an anchor sheath; (ii) at least one anchor passing suture looped through the anchor sheath; and (iii) at least one anchor deployment suture attached to the anchor sheath. An anchor passing suture may be a leader suture that pulls the anchor sheath into a hole such as a pilot hole. An anchor deployment suture may be cinching suture that deploys the anchor sheath from a relaxed, first position to a bunched up, second position. In some implementations, an anchor passing suture may slide relative to the anchor sheath of the soft suture anchor. In some implementations, an anchor deployment suture may slide relative to the anchor sheath of the soft suture anchor. The soft suture anchor may include one or more shuttle/pull devices. In some implementations, both the anchor passing and deployment sutures may extend in about a similar direction and orientation relative to an axis of the sheath.

The novel soft suture anchor design and technique eliminate the likelihood of anchor pullout from the pilot hole and obviates the need for impaction to achieve implantation.

Methods of knotless tissue repairs are also disclosed. In some implementations, a surgical construct provides knotless tissue to tissue fixation, for example, knotless fixation of soft tissue (ligament, tendon, graft, etc.) to bone without any knot formation and in a simple and fast manner. The tensionable repairs with all-suture anchors facilitate drill tunnel holes smaller than those of traditional techniques that utilize hard body anchors and require larger bone holes. The novel technique also obviates the need for anchor impaction to secure anchor insertion into bone.

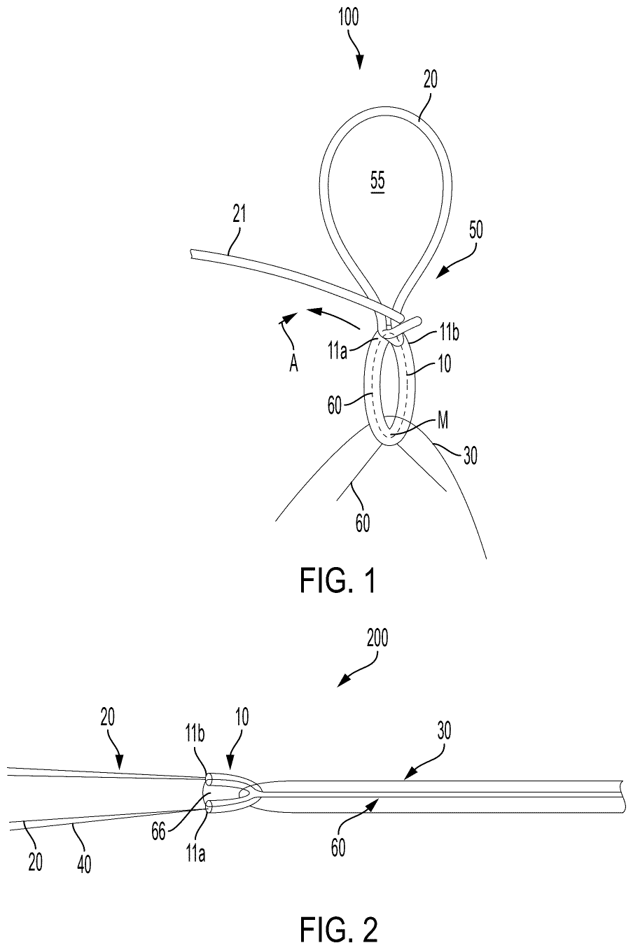

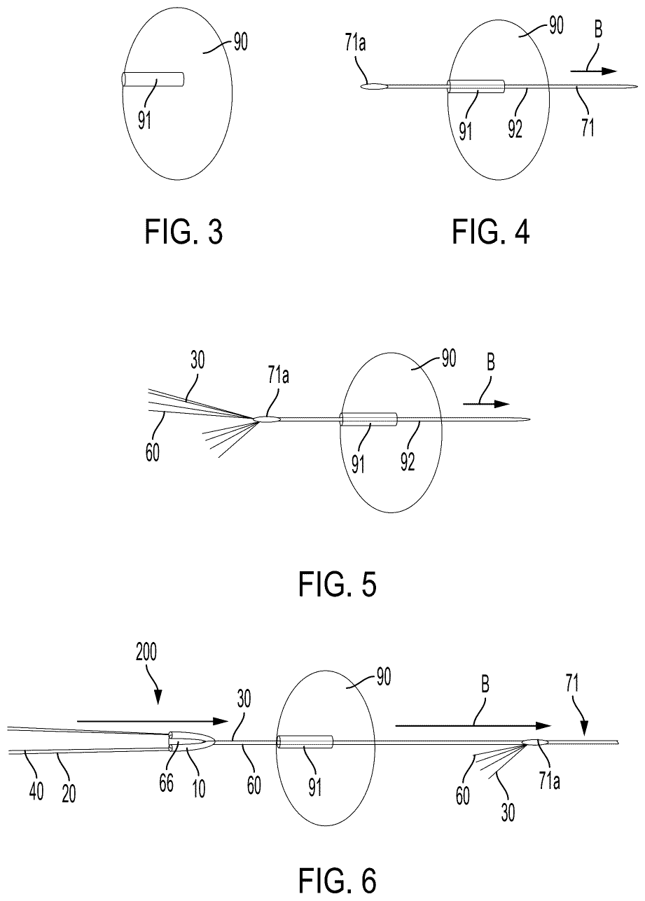

Referring now to the drawings, where like elements are designated by like reference numerals, FIGS. 1, 2 and 14 illustrate exemplary fixation devices 100, 200. FIGS. 3-13 illustrate steps of exemplary soft tissue repair 101 with exemplary fixation device 200.

Fixation device 100, 200 may be a soft anchor. Fixation device 100, 200 may be a soft suture anchor construct. Fixation device 100, 200 may be a surgical construct. Fixation device 100, 200 may be a surgical system. Fixation device 100, 200 may be a tensionable, knotless, self-locking, surgical construct.

As detailed below, fixation device 100, 200 is an implantable device made completely of suture or sutures that achieve fixation in bone without a separate anchoring body. The design enables fixation by deployment into bone with one or more suture tails remaining outside the bone for device attachment, pulling, bunching, tensioning and/or alternative usage.

FIG. 1 illustrates a schematic view of surgical construct 100 in the relaxed, undeployed position. Construct 100 includes (i) a soft anchor 50 with cannulated sheath 10 and at least one flexible strand 20; (ii) at least one passing strand 30; and (iii) at least one deployment strand 60. The at least one flexible strand 20 may be a repair suture. The at least one passing strand 30 may be an anchor passing suture or leader suture. The at least one deployment strand 60 may be an anchor deployment suture or cinch suture. Construct 100 may also include a shuttle/pull device 40 (shown in FIG. 2) to aid in passing of the flexible strand 20 through the cannulated sheath 10 to allow formation of a splice and at least one adjustable, knotless, closed, continuous loop.

Cannulated sheath 10 of soft anchor 50 can be in the form of a tubular sleeve or tubular member with a body made of a flexible material, such as a braided, woven, or knitted structure made of yarns, fibers, filaments, sutures or similar materials, or combinations of these materials. In some implementations, sheath 10 may be constructed of ultra-high molecular weight polyethylene (UHMWPE). In some implementations, sheath 10 may be constructed of UHMWPE and polyester. In some implementations, sheath 10 may be constructed of UHMWPE with an elastic component. In some implementations, sheath 10 may be constructed of polyester suture material with an elastic component. The elastic component may be elastane. The elastic component may be incorporated into the sheath via braiding, weaving, and/or knitting. In some implementations, an elastic sheath may include a combination of elastic, polyester, and UHMWPE all braided in a tubular jacket. An elastic sheath may consist essentially of elastane, polyester, and UHMWPE. The elastic component provides elasticity while the other components provide strength and limit the elongation of the suture.

Sheath 10 can also be any tubular or non-tubular structure having a lumen along its length. The lumen can have a constant diameter. The lumen can have a varying diameter along its length.

Tubular body of sheath 10 extends between opposing ends 11a, 11b. The opposing ends 11a, 11b can be open or closed ends. The tubular body establishes the bore or lumen that extends between the opposing ends 11a, 11b. One or more flexible strands 20 (flexible coupler 20; suture 20; repair suture 20) may be passed through at least a portion of bore of the sheath 10 and through the body of the sheath 10 with an exemplary free end 21 exiting one of the ends 11a, 11b, or at a location close to the one of the free ends 11a, 11b. Flexible strand 20 located within the body of the sheath 10 may be fixed relative to the sheath (or affixed to the sheath 10), for example, by being stitched to the sheath 10, or may slide through the sheath 10.

FIG. 1 illustrates an exemplary-only implementation of soft anchor 50 with flexible strand 20 passing through the sheath 10 and with only one free end 21 exiting the sheath 10. Tensioning of the free end 21 may be conducted by pulling end 21 in the direction of arrow A to allow loop 55 to decrease its length and diameter and tension the final repair. Loop 55 may be an adjustable, flexible, tensionable, closed, knotless loop formed around soft tissue to be secured to the bone, and as detailed below.

Soft anchor 50 may also include a shuttle/pull device 40 (shown in FIG. 2) to aid in the formation of at least one loop 55 and corresponding splice. Shuttle/pull device 40 may be a suture passing instrument, a suture passer, a shuttle link, a FiberLink™, a nitinol loop. Shuttle/pull device 40 includes an eyelet/loop for passing flexible coupler 20. If more than one shuttle/pull devices 40 are provided, the shuttle/pull devices can extend through the sheath 10 in similar or different directions and/or orientations and/or locations. The at least one loop 55 mat be an adjustable, knotless, closed, self-cinching, continuous loop with an adjustable perimeter and/or length.

In some implementations, soft suture anchor 50 may be a knotless FiberTak® soft anchor that may be a single-loaded, double-loaded or multiple-loaded anchor. In some implementations, soft suture anchor 50 may be a knotless FiberTak® soft anchor that may be a hybrid FiberTak® soft anchor with a shuttle/pull device that acts both as a shuttling suture and as a repair suture. In some implementations, soft suture anchor 50 may be a knotless FiberTak® soft anchor with a single repair suture that is furcated, bifurcated, trifurcated, etc. In some implementations, soft suture anchor 50 may be a knotless FiberTak® soft anchor with a plurality of repair sutures 20 and a plurality of corresponding shuttle/pull devices 40. The number of the repair sutures 20 and the number of the shuttle/pull devices 40 may be similar or different. Details of an exemplary soft suture anchor with a soft anchor sleeve (sheath or tubular member) and flexible shuttling strands are set forth, for example, in U.S. application Ser. No. 15/998,516 entitled “Methods of Tissue Repairs” filed on Aug. 16, 2018 (issued as U.S. Pat. No. 10,849,734 on Dec. 1, 2020), the disclosure of which is incorporated by reference in its entirety herein. The flexible coupler(s) and the one or more shuttle/pull devices can extend through the sleeve in similar or different directions and/or orientations and/or locations.

FIG. 1 also illustrates two additional flexible couplers 30, 60. Flexible coupler 30 may be an anchor passing suture or a leader suture. Flexible coupler 30 may be looped or passed over the sheath 10, as shown in FIG. 1. As detailed below, leader suture 30 is used to pull anchor body 10 and soft anchor 50 into a pilot hole which has been pre-prepared in the bone. The pilot hole may consist of a socket (such as an exemplary 2.6 mm socket) at the fixation site in the near cortex communicating with a smaller diameter transosseous tunnel (placed from the bottom of the socket, through the far cortex) and without deploying the anchor.

Flexible coupler 60 may be an anchor deployment suture or a cinch suture. Anchor deployment suture 60 may be a “cinch” suture that is spliced into (or enters) the sheath 10 at about the sheath midpoint M, is routed out the open end 11b of one side of the sheath 10, back in the opposite open end 11a of the sheath 10, and exiting again at the midpoint M, adjacent to where it was initially spliced in. Passage of deployment suture 60 through the sheath 10 and ends 11a, 11b forms a loop 66 (FIG. 2). Loop 66 may be an adjustable, knotless loop. In some implementations, loop 66 may be a closed, continuous loop with an adjustable diameter and length.

In some embodiments, deployment suture 60 may be pierced into the apex or middle M of the sheath 10, exiting the cannulation of the sheath at end 11b or around the end 11b, and re-entering the cannulation of the other end 11a of the sheath or around the end 11a, and exiting the sheath again through (or adjacent to) the point M where it is initially pierced.

As detailed below, when deployment suture 60 is tensioned, the length of loop 66 decreases and the anchor sheath 10 deploys from a relaxed, first position to a “bunched,” deployed, second position. Once the anchor 50 has been deployed, one limb of the lead suture 30 and one limb of the deployment suture 60 may be pulled or shuttled to remove both the passing and deployment sutures 30, 60. Alternatively, the deployment suture 60 may be employed for additional surgical procedures such as knot tying, use with additional fixation devices such as hard-body anchors, soft-body anchors, etc. In some implementations, the passing suture 30 and deployment suture 60 may have different colors for improved intraoperative suture identification and management.

Reference is now made to FIG. 2 which illustrates a schematic view of exemplary surgical construct 200. Construct 200 is about similar to construct 100 detailed above in that soft anchor 50 also includes a cannulated sheath 10 with two additional strands 30, 60. However, construct 200 is illustrated with partial view of flexible repair suture 20 and shuttle/pull device 40 before the formation of knotless loop 55 shown in FIG. 1. As shown in FIG. 2, the at least one flexible strand 20 extends in a first direction, and the deployment suture 60 and passing suture 30 extend in a second direction. In some implementations, the first direction may be different from the second direction. The first direction may be about opposite the second direction.

Although the embodiments and implementations detailed above have been described with reference to a soft anchor 50 provided with one repair suture 20, 21, one anchor passing suture 30, and one anchor deployment suture 60, it must be understood that the disclosure is not limited to this exemplary-only implementation and contemplates soft anchors provided with any number of repair sutures, anchor passing sutures 30 and deployment sutures 60, depending on the specifics and characteristics of the surgical repair and the surgeon's preferences.

Reference is now made to FIGS. 3-14 which illustrate schematic steps of exemplary soft tissue repair 101 that allows tissue 80 to be attached to tissue 90 with exemplary surgical construct 200 of FIG. 2. The all-suture anchor repair 101 is a tensionable, knotless repair that facilitates smaller drill tunnel holes, fewer surgical steps, and increased bunching of sheath 10 within a bone socket/tunnel.

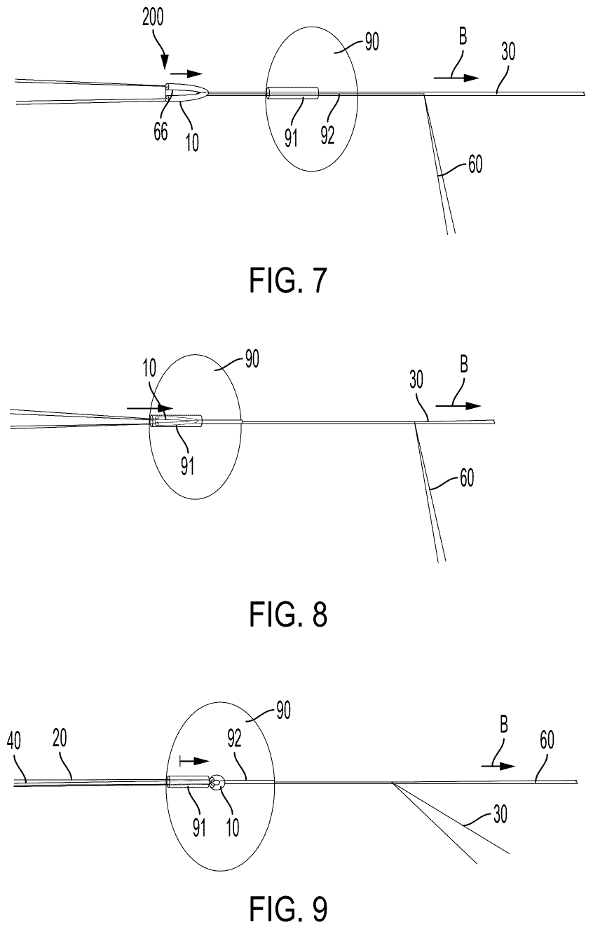

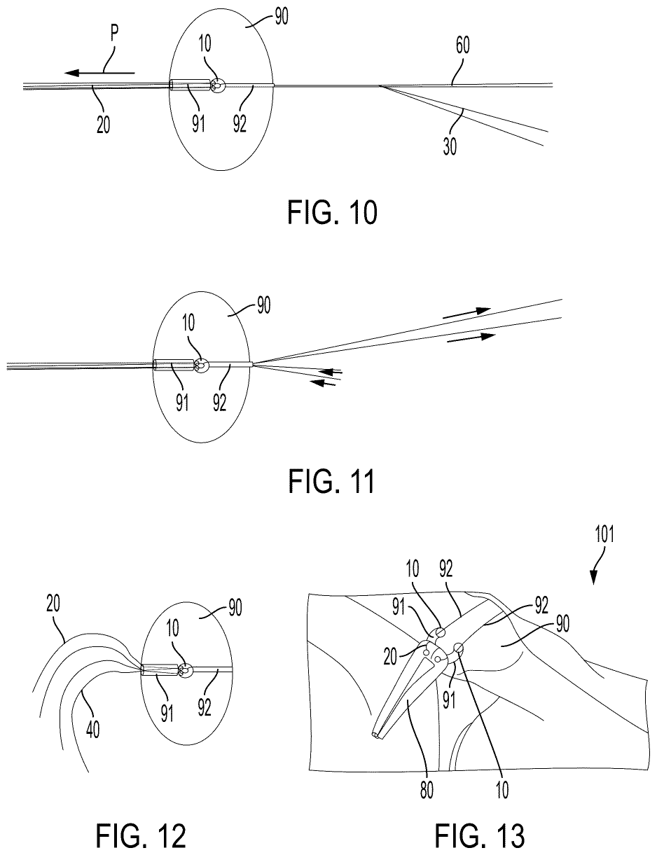

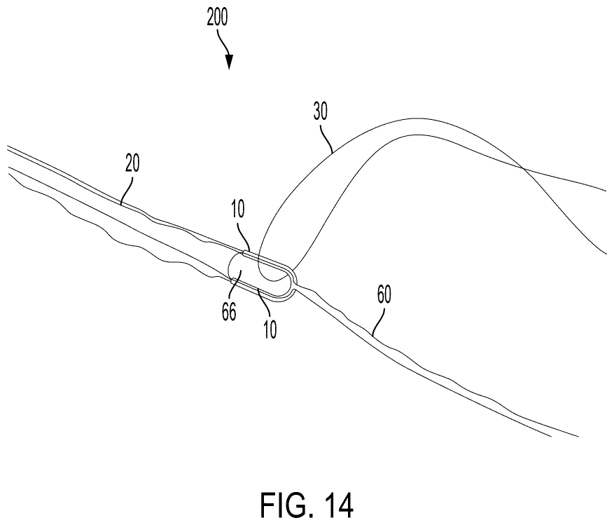

The implementation below will be detailed with reference to a specific repair, the medial patellofemoral ligament (MPFL) repair of the knee where the surgeon utilizes FiberTak® anchors and constructs 100, 200 to secure a suture tape (FiberTape® suture tape) that acts like a “brace” to support the repaired ligament, providing additional stability during the healing process; essentially augmenting the primary MPFL repair by increasing the area of tissue approximation around the repair site. However, soft anchor 100, 200 is configured for use in various soft tissue repairs or fixations and can be fixated inside bone or partially inside bone for attaching tissue (e.g., ligament, tendon, graft, etc.) to bone. For example, soft anchor 100, 200 may be used in conjunction with a variety of orthopedic surgical repairs, including but not limited to rotator cuff repairs, Achilles tendon repairs, patellar tendon repairs, ACL/PCL reconstructions, hip and shoulder reconstructions, among many others. The fixation can be on or over bone.
FIG. 3: Drill a pilot hole 91 in patella 90; pilot hole 91 may be an exemplary 2.6 mm pilot hole.
FIG. 4: From the bottom of the pilot hole 91, drill a smaller diameter tunnel 92. An exemplary 1.6 mm diameter tunnel (transosseous tunnel) may be drilled with a k wire 71 provided with a wire loop 71a out the far side of the patella 90.
FIG. 5: Load anchor passing suture 30 and anchor deployment suture 60 in the k wire loop to shuttle both sutures 30, 60 through the patella 90, from a proximal side to a distal side and in the direction of arrow B.
FIG. 6: Pull k wire 71 though lateral side of patella 90 in the direction of arrow B, exposing passing and deployment sutures 30, 60 of construct 200 and dispose of the k wire 71.
FIG. 7: Using the anchor passing suture 30, pull the anchor 50 into the pilot hole 91.
FIG. 8: Pull the anchor passing suture limbs 30 to pull anchor body 10 all the way to the bottom of the pilot hole 91. Features may be included on the sutures or outriggers developed as visual identifiers to reference anchor depth.
FIG. 9: With anchor body 10 firmly at the bottom of the pilot hole 91, tension the anchor deployment suture 60 to “bunch up” the anchor body 10 (cannulated sheath 10) at the bottom of the pilot hole 91. By pulling the anchor deployment suture 60 in the direction of arrow B, the loop 66 formed by anchor deployment suture 60 becomes smaller and allows bunching or deployment of the sheath 10 from an undeployed or relaxed position (first position) to a deployed or bunched up position (second position).
FIG. 10: Following manual deployment of the anchor 50, firmly pull the FiberTak® repair suture(s) 20 in the direction of arrow P to confirm that the anchor has fully deployed or “set” in the bone 90. If displacement occurs, previous steps may be repeated to pull the anchor back into the pilot hole and redeploy it.
FIG. 11: Once anchor deployment has been confirmed, pull one limb of each of the anchor shuttle suture 30 and anchor deployment suture 60 to pull them out of the anchor 50 and out of the patella 90.
FIG. 12: Proceed to complete the repair by using the FiberTak® repair sutures 20 and shuttle/pull device 40.
FIG. 13: Exemplary repair 101 with two constructs 100, 200 of the present disclosure employed for medial patellofemoral ligament (MPFL) repair of the knee is shown in FIG. 13. Graft 80 is attached to the femoral socket in a double bundle reconstruction with the aid of repair sutures 20 of surgical constructs 100, 200 secured within two pilot holes 91 in patella 90.
FIG. 14 illustrates an additional view of the surgical construct 200 of FIG. 2.

The implant 100, 200 detailed above is a manual-bunching knee FiberTak® construct. The new anchor design allows the anchor to be pulled into a pre-prepared pilot hole using a lead suture 30, then manually deployed or “bunched” using a cinch suture 60 that is routed through the anchor sheath 10. This design and technique lessen the likelihood of anchor pullout (anchor pullout being due to the anchor not bunching in the pilot hole 91) and obviates the need for impaction to achieve implantation. Current standard implantation technique for knee FiberTak® anchors uses impaction to drive the anchor into bone or a prepared pilot hole in the bone, relying on friction of the anchor sheath to achieve anchor deployment or “bunching.” The disclosed technique eliminates the need for impaction of anchors in bone, eliminating therefore anchor loosening or pull out from bone socket, improper anchor placement, bone fractures, infections, and any additional repair complications.

The implant 100, 200 may employ a FiberTak® anchor with the addition of (1) a leader suture 30 passed through the loop of the anchor body and used to pull the anchor into a pilot hole which has been pre-prepared in the bone consisting of (in the case of knee) a 2.6 mm socket at the fixation site in the near cortex, with a smaller diameter transosseous tunnel, placed from the bottom of the 2.6 mm socket, through the far cortex, without deploying the anchor and (2) a “cinch” suture 60 that is spliced into the sheath at the sheath midpoint, routed out the open end of one side of the sheath, back in the opposite open end of the sheath, and exiting again at the midpoint, adjacent to where it was initially spliced in, which when tensioned, deploys the anchor by “bunching” the sheath. Once the anchor is deployed, one limb of both the lead suture 30 and the deployment suture 60 can be pulled or shuttled to remove them.

By the present disclosure, the implant 100, 200 is pulled into a pilot hole rather than being impacted using a driver. The implant 100, 200 is manually deployed or “bunched” using the cinch suture 60 rather than relying on deployment by friction of the sheath against the wall of the pilot hole.

If more than one flexible coupler 20, anchor passing suture 30 and anchor deployment suture 60 are employed, the plurality of flexible couplers, passing sutures and deployment sutures can reside within and around the cannulated sheath 10 independent from each other. The flexible couplers may be similar to each other or different from each other. The deployment sutures may be similar to each other or different from each other. In some implementations, the flexible coupler 20 can be bifurcated or furcated from the sheath 10 to provide a plurality of loops.

In some implementations, the flexible coupler 20 may come out of the cannulated sheath 10 and be braided to a smaller diameter round suture which may be passed back through the sheath. The braided suture may be furcated/branched/forked (for example, bifurcated or trifurcated, etc.) to provide a plurality of passing sutures. In some implementations, the end of flexible coupler 20 may be a tapered end. In some implementations, flexible coupler 20 may be a single strand suture. In some implementations, flexible coupler 20 may be a single braided suture.

The constructs, systems, and assemblies of the present disclosure may be employed in numerous knotless soft tissue repairs and fixations, for example, fixation of soft tissue to bone. In some implementations, a surgical construct 100, 200 may provide first tissue to second tissue fixation, for example, fixation of soft tissue 80 (ligament, tendon, graft, etc.) to bone 90 in a simple and fast manner.

The fixation devices 100, 200 detailed above are soft anchors formed of “soft” materials, such as suture materials, yarns, fibers, filaments, strings, fibrils, strands, etc., or any combination of such materials. The soft materials may be synthetic or natural materials, or combinations of synthetic and natural materials, and may be bio-degradable or non-degradable, and may be elastic or non-elastic within the scope of this disclosure. These soft materials confer the ability to be inserted into bone sockets/holes/tunnels and bunch together, collapse, expand and/or change shape to fixate within the socket/hole/tunnel. In some implementations the soft anchor 100, 200 is made exclusively of soft, suture-based materials. Soft anchor 100, 200 may be an “all-suture” construct.

Soft anchor 100, 200 is configured for use in various soft tissue repairs or fixations and may be fixated inside bone or over bone for attaching tissue (e.g., ligament, tendon, graft, etc.) to bone. For example, the soft anchor 100, 200 can be used in conjunction with a variety of orthopedic surgical repairs, including but not limited to rotator cuff repairs, Achilles tendon repairs, patellar tendon repairs, ACL/PCL reconstructions, hip and shoulder reconstructions, among many others. The fixation can be on or over bone.

A soft suture anchor 100, 200 comprises: (i) a cannulated sheath 10 having a length, a first end 11a, and a second end 11b, and at least one flexible coupler 20 attached to the cannulated sheath 10; (ii) a passing suture 30 attached to the sheath 10; and (iii) a deployment suture 60 passing through the sheath 10. The at least one flexible coupler 20 extends in a first direction and the deployment suture 60 extends in a second direction. The first direction may be different from the second direction. The first direction may be opposite the second direction. At least one of the flexible coupler 20, passing suture 30 and deployment suture 60 may be formed of elastic suture. At least one of the passing suture 30 and deployment suture 60 may be completely removed from the suture anchor 100, 200.

Methods of soft tissue repair which do not require tying of knots and allow adjustment of both the tension of the suture and the location of the tissue with respect to the bone, while providing a self-locking mechanism, are disclosed.

A method of knotless tissue repair 101 comprises inter alia the steps of: (i) inserting a tubular sheath 10 of a suture anchor 50 of a suture anchor construct 100, 200 into a pre-formed hole 91 in a first tissue 90; and (ii) securing the tubular sheath 10 by manually deploying the tubular sheath within the pre-formed hole 91. The method may further comprise securing a deployment suture 60 to the tubular sheath; pulling on a passing suture 30 to pull the suture anchor 50 and insert the suture anchor 50 within the pre-formed hole 91; and pulling on the deployment suture 60 to manually bunch up the tubular sheath 10 within the pre-formed hole 91. The method may further comprise forming at least one adjustable, knotless, tensionable loop 55 around tissue 80 to be attached to the tissue 90 with the flexible coupler 20 and a shuttle/pull device 40. The method may further comprise employing the flexible coupler 20 for additional surgical procedures. The additional surgical procedures may include passing the flexible coupler 20 through or around tissue 80 multiple times; and/or employing the flexible coupler 20 with additional fixation devices. The additional fixation devices can be any of a knotless anchor, a hard body anchor, or a soft anchor.

The knotless tissue repair and reconstruction can be any of small joint repairs, rotator cuff repairs, Achilles tendon repair, patellar tendon repair, ACL/PCL reconstruction, hip and shoulder reconstruction procedures, and applications for elastic suture used in or with suture anchors. The surgical constructs and repair methods of the present disclosure can be employed in tissue repairs that may not involve knot tying, for example, for use with suture anchors (such as PushLock® and/or SwiveLock® suture anchors) or for knotless arthroscopic suture repairs (such as knotless single row rotator cuff repair, or SpeedBridge™ repairs using no knots and only suture passing steps), among many others.

Any of sheath 10, repair suture 20, passing suture 30 and deployment suture 60 can include a flexible material, for example, multifilament, braided, knitted, woven suture, or can include fibers of ultrahigh molecular weight polyethylene (UHMWPE) or the FiberWire® suture (disclosed in U.S. Pat. No. 6,716,234, the disclosure of which is hereby incorporated by reference in its entirety herein).

Repair suture 20, passing suture 30 and deployment suture 60 may be any flexible coupler or flexible strand and may be formed of various flexible materials and strands such as round suture, flat suture, ribbon, or flat tape (for example, suture tape) or combination of suture and tape. Exemplary materials may include suture, silk, cotton, nylon, polypropylene, polyethylene, ultrahigh molecular weight polyethylene (UHMWPE), polyethylene terephthalate (PET), and polyesters and copolymers thereof, or combinations thereof. Repair suture 20, passing suture 30 and deployment suture 60 may have cross-sections of various forms and geometries, including round, oval, rectangular, or flat, among others, or combination of such forms and geometries.

In some implementations, repair suture 20, passing suture 30 and deployment suture 60 may be formed of a high strength suture material such as FiberWire® suture, sold by Arthrex, Inc. of Naples, Fla., and described in U.S. Pat. No. 6,716,234, the disclosure of which is incorporated by reference herein. FiberWire® suture is formed of an advanced, high-strength fiber material, namely ultrahigh molecular weight polyethylene (UHMWPE), sold under the tradenames Spectra® (Honeywell International Inc., Colonial Heights, Va.) and Dyneema® (DSM N.V., Heerlen, the Netherlands), braided with at least one other fiber, natural or synthetic, to form lengths of suture material. Repair suture 20, passing suture 30 and deployment suture 60 may be braided or multi-filament suture such as FiberTape® suture tape (as disclosed in U.S. Pat. No. 7,892,256, the disclosure of which is incorporated in its entirety herewith). Repair suture 20, passing suture 30 and deployment suture 60 may include elastic material. Repair suture 20, passing suture 30 and deployment suture 60 may consist essentially of elastic suture.

Repair suture 20, passing suture 30 and deployment suture 60 can be also formed of a stiff material, or combination of stiff and flexible materials, particularly for the regions of the coupler that may need to be passed/spliced through the body of the coupler and depending on whether they are employed with additional fixation devices.

Various structural elements of surgical construct 100, 200 including repair suture 20, passing suture 30 and deployment suture 60 may be visually coded, making identification and handling of the sheath and suture legs simpler. Easy identification of suture in situ is advantageous in surgical procedures, particularly during arthroscopic surgeries, endoscopic and laparoscopic procedures.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed:

1. A soft anchor comprising:
a cannulated sheath having a length, a first end, a second end, and at least one repair suture attached to the cannulated sheath;
a first flexible strand removably attached to the cannulated sheath; and
a second flexible strand attached to the cannulated sheath and configured to slide through at least a portion of the cannulated sheath.

2. The soft anchor of claim 1, wherein the second flexible strand has a first pull direction and the at least one repair suture has a second pull direction, wherein the first pull direction is different from the second pull direction.

3. The soft anchor of claim 2, wherein the first pull direction is about opposite the second pull direction.

4. The soft anchor of claim 2, wherein pulling the second flexible strand in the first pull direction deploys and bunches up the cannulated sheath.

5. The soft anchor of claim 1, wherein the second flexible strand is removably attached to the cannulated sheath.

6. The soft anchor of claim 1, wherein the second flexible strand has a first end and a second end and wherein the second flexible strand is configured to extend through the at least a portion of the cannulated sheath and along its length, and wherein the first end and the second end exit the cannulated sheath at a location along the length of the cannulated sheath.

7. The soft anchor of claim 6, wherein the location is at about a mid-length of the cannulated sheath.

8. The soft anchor of claim 1, wherein the cannulated sheath is a tubular sheath secured into bone, wherein a free end of the at least one repair suture passes through the tubular sheath at least once to form a splice and an adjustable, flexible, tensionable, knotless loop around soft tissue to be secured to the bone.

9. The soft anchor of claim 1, wherein the soft anchor is an all-suture soft anchor and the at least one repair suture is round suture.

10. The soft anchor of claim 1, wherein the cannulated sheath is elastic.

11. The soft anchor of claim 1, wherein the cannulated sheath further comprises a shuttle/pull device.

12. The soft anchor of claim 1, wherein the at least one repair suture is furcated into a plurality of repair sutures.

13. A method of tissue repair, comprising:
inserting a cannulated sheath of a soft suture anchor into a first tissue; and
securing the cannulated sheath within the first tissue by manually deploying the cannulated sheath.

14. The method of claim 13, wherein the soft suture anchor includes at least one repair suture attached to the cannulated sheath, a first flexible coupler looped around the cannulated sheath, and a second flexible coupler attached to the cannulated sheath, the second flexible coupler being configured to slide through the cannulated sheath, the second flexible coupler including two opposing tails, and wherein the method further comprises:
pulling the first flexible coupler to insert the cannulated sheath into a pre-formed hole in the first tissue; and
pulling the two opposing tails of the second flexible coupler to secure the cannulated sheath within the pre-formed hole.

15. The method of claim 14, further comprising discarding at least one of the first flexible coupler and the second flexible coupler.

16. The method of claim 14, wherein pulling the first flexible coupler and pulling the second flexible coupler are conducted in a same direction.

17. The method of claim 14, wherein pulling the two opposing tails of the second flexible coupler changes the cannulated sheath from a first configuration to a second configuration.

18. The method of claim 17, wherein the second configuration is a bunched-up configuration.

19. The method of claim 14, further comprising forming, with the at least one repair suture, a knotless loop around a second tissue to be attached to the first tissue.

20. The method of claim 19, wherein the knotless loop is a closed, continuous, tensionable, adjustable loop formed around, through, or over the second tissue.

21. The method of claim 19, wherein the first tissue is bone, and the second tissue is soft tissue.

22. The method of claim 19, wherein the first tissue is bone and the second tissue is tendon or ligament.

23. The method of claim 13, wherein the tissue repair is medial patellofemoral ligament repair.
